(19) United States
(12) Patent Application Publication
(10) Pub. No.: US 2019/0336330 A1
(43) Pub. Date: Nov. 7, 2019

(54) Head-cooling cap

(71) Applicant: Thomas J. Hickey, North Palm Beach, FL (US)

(72) Inventor: Thomas J. Hickey, North Palm Beach, FL (US)

(21) Appl. No.: 16/501,977

(22) Filed: Jul. 15, 2019

Related U.S. Application Data
(63) Continuation of application No. 14/937,045, filed on Nov. 10, 2015.
(60) Provisional application No. 62/077,430, filed on Nov. 10, 2014.

(51) Int. Cl. A61F 7/02

(52) U.S. Cl. CPC A61F 7/02; A61F 2007/0005; A61F 2007/0244; A61F 2007/0271

(57) ABSTRACT

A head-cooling cap for controlling swelling caused by any head injury is provided. The head-cooling cap includes a flexible, preferably adjustable cap-shaped main body with a plurality of inner cavities containing a cooling substance. The main body can be built in a three-dimensional cap shape or, alternatively, be deformable to adopt a three-dimensional cap shape. When the head-cooling cap is placed on a user's head, the cooling substance, which has been kept at an adequate cold temperature, will cool the head and control any swelling caused by a traumatic head injury. When not placed on a user's head, the head-cooling cap can be conveniently stored in a refrigerator or freezer to prepare it for further use.

### CROSS-REFERENCE TO RELATED APPLICATION

This application claims the benefit of co-pending U.S. Provisional Patent Application Ser. No. 62/077,430, filed Nov. 10, 2015, which is incorporated herein in its entirety.

### FIELD OF THE INVENTION

The present invention relates generally to medical devices, and more particularly, to a head-cooling cap that can be placed on a person's head to cool the person's head and thus help reducing swelling caused by any head injury.

### BACKGROUND OF THE INVENTION

Head injuries are injuries that result in trauma to the scalp, skull, or brain. Head injuries generally fall into two categories: external injuries and internal injuries. External injuries usually involve the scalp while internal injuries may involve the skull, the blood vessels within the skull, or the brain. Head injuries can be caused by a variety of reasons including, for example, slip and falls, traffic accidents, physical assaults, accidents at home, work, outdoors or while playing sports.

Most head injuries are minor because the skull protects the brain. However, traumatic head injuries, especially sport related head injuries, can be a major cause of disability or even death. Sport related head injuries may be caused by, for example, blows received by a boxer during a boxing match or hits and collisions received by a football player during a football game.

All head injuries may cause swelling or edema, which may increase the pressure inside the skull and cause the brain to compress against the skull. This pressure and compression could reduce or cut off the blood and oxygen supply to the brain, which when untreated or not treated timely could lead to permanent brain damage or death.

There are several treatments for brain edema. The goal of all the treatments is to help the brain receiving enough blood and oxygen to remain healthy while the swelling is relieved and the underlying causes are treated. However, most of the treatments, such as oxygen therapy, IV fluids, medication, ventriculostomy, and surgery, require attending hospital facilities or at least receiving adequate professional care, which are typically not available immediately after an injury.

Hypothermia, which refers to lowering body temperature to below what is normally required, helps relieve swelling and allows the brain to heal. Although general hypothermia may be difficult to perform correctly and may potentially be dangerous, localized hypothermia to the area where an injury occurred can help to control swelling caused by trauma and the rate of blood circulation to the brain.

Normally, localized hypothermia is achieved by applying ice onto the injured body part. The ice is generally concealed inside a bag or cloth to prevent ice burning of the skin. As those skilled in the art have experienced, in dependence of the body part and/or the situation in which ice needs to be applied, handling ice can result extremely inconvenient and cumbersome. For instance, it is particularly difficult to apply ice to a person's head, as the ice bag or ice-holding cloth often slips towards the person's face and ears, causing discomfort and strongly interfering with the person's eyesight, hearing or breathing.

Accordingly, there is an established need for a convenient and effective head-cooling cap that can be placed on a person's head immediately after a head injury to cool the person's head and thus help reducing swelling caused by trauma.

### SUMMARY OF THE INVENTION

The present invention is directed to a convenient and effective head-cooling cap that can be placed on a person's head immediately after a head injury and that is capable of cooling the person's head to help controlling swelling caused by trauma (such as blows received when practicing or competing in boxing or other active sports) and thus maintaining blood and oxygen supply to the brain. The cap can be rapidly and conveniently fit onto and removed from the person's head, causing no discomfort to the user. For instance, a boxer could wear the head-cooling cap for brief periods of time between rounds, and control swelling caused by blows to the head received during the rounds. The head-cooling cap could also be used in hospitals, emergency rooms, or ambulances to rapidly cover a patient's head in the event of head trauma and swelling.

The head-cooling cap is formed of several flexible, plastic portions, integrally formed with one another, or attached to each other by stitching, heat welding, or other applicable fasteners. The portions can include inner cavities housing a cooling or freezing substance, such as a cooling or refrigerant gel or liquid. In order to prepare the cap for use, the cap can be stored at freezing or cold temperatures for a sufficient amount of time to cause the cooling or refrigerant substance to become adequately cold or frozen. The cap is then placed on a user's head in order to reduce swelling and/or pain. For instance, wearing the cap for brief periods of time will allow an athlete who has received blows to the head to immediately keep down swelling and assist blood flow. The cap can be manufactured in different shapes and sizes for users to choose from. Alternatively, the cap can be made adjustable so as to adapt to different shapes and sizes of heads.

In a first implementation of the invention, a head-cooling cap for controlling swelling caused by any head injury comprises a flexible main body configured to present a cap shape for fitting onto a user's head, said main body comprising a plurality of cooling elements, each cooling element of the plurality of cooling elements comprising a cooling substance housed within an inner cavity of the main body.

In a second aspect, the inner cavity of each cooling element can be formed between two layers of flexible material that form the flexible main body.

In another aspect, the main body can include a concave top portion comprising at least one cooling element of the plurality of cooling elements, and a side portion extending from a bottom end of the concave top portion, the side portion comprising at least one cooling element of the plurality of cooling elements. Thus, the cap not only cools the top half of the head, but can also cool any one of the neck and one or both the ears, in dependence of the extension of the side portion. For instance, the side portion can include two opposed ear portions for covering a user's ear, each ear portion comprising at least one respective cooling element of the plurality of cooling elements. Each ear portion can optionally include a through hole configured to be arranged in registration with an opening of an ear canal of a user wearing the head-cooling cap, to facilitate a user's hearing while wearing the head-cooling cap. To maximize cooling of the ears, each ear portion can include a respective cooling element partially or fully surrounding the through hole. Alternatively or additionally, the side portion can include a neck portion for covering a back of a neck of a user, the neck portion comprising at least one cooling element.

In another aspect, the head-cooling cap includes at least one elastic adjuster to adjustably fit the flexible main body to a user's head, allowing the device to be used by users of all ages and head sizes. For instance, the elastic adjuster can extend along the neck portion and ear portions. Preferably, the elastic adjuster is arranged above the neck portion and ear portions.

In another aspect, the main body can be permanently formed in the cap shape. For instance, the main body can include several separate body portions connected together by at least one of stitching and welding to form the cap shape.

In another aspect, the main body can include a flexible front portion, a flexible rear portion, and opposed flexible lateral portions, the front, rear and lateral portions respectively comprising at least cooling element of the plurality of cooling elements. The front, rear and lateral portions extend at a proximal end thereof from a crown portion, the front, rear and lateral portions being selectively deformable to adopt different configurations. In a first, flat configuration, the front, rear and lateral portions extend radially from the crown portion, facilitating storage, transportation and packaging of the head-cooling cap. In a second configuration, the front, rear and lateral portions are curved inward to form a cap shape for fitting onto a user's head. Ear portions can be provided at respective distal ends of the lateral portions. Alternatively or additionally, a neck portion can be provided at a distal end of the rear portion. The head-cooling cap further comprises at least one fastener for retaining the front, rear and lateral portions in the second configuration. The at least one fastener can include two straps extending from the rear portion, the straps being disconnectably attachable to the front portion. Preferably, the straps extend from opposite ends of a distal section of the rear portion and are disconnectably attachable to a distal section of the front portion.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents an isometric view of a first exemplary embodiment of a head-cooling cap in accordance with the present invention, comprising several top cooling elements, two ear cooling elements, and one rear side cooling element (for the back of the neck);

FIG. 2 presents a side elevation view of the head-cooling cap of FIG. 1;

FIG. 3 presents a rear elevation view of the head-cooling cap of FIG. 1;

FIG. 4 presents an enlarged, partial cross-sectional view of the head-cooling cap of FIG. 1, taken along plane 4-4 indicated in FIG. 3, the cross-section revealing an inner cavity housing a cooling gel;

FIG. 5 presents a front elevation view of the head-cooling cap of FIG. 1, before being placed on a user's head;

FIG. 6 presents a front elevation view of the head-cooling cap of FIG. 1, after being placed on a user's head;

FIG. 7 presents an isometric view of a second exemplary embodiment of a head-cooling cap in accordance with the present invention, which comes in an initially flat format and can be deformed into a three-dimensional cap form; this embodiment includes rear attachment straps, configured to fasten to a front portion by hook-and-loop fasteners; and

FIG. 8 presents an isometric view of the cap of FIG. 7, shown deformed into the three dimensional cap form, prior to attaching the strap to the front portion via the hook-and-loop fasteners.

Like reference numerals refer to like parts throughout the several views of the drawings.

### DETAILED DESCRIPTION

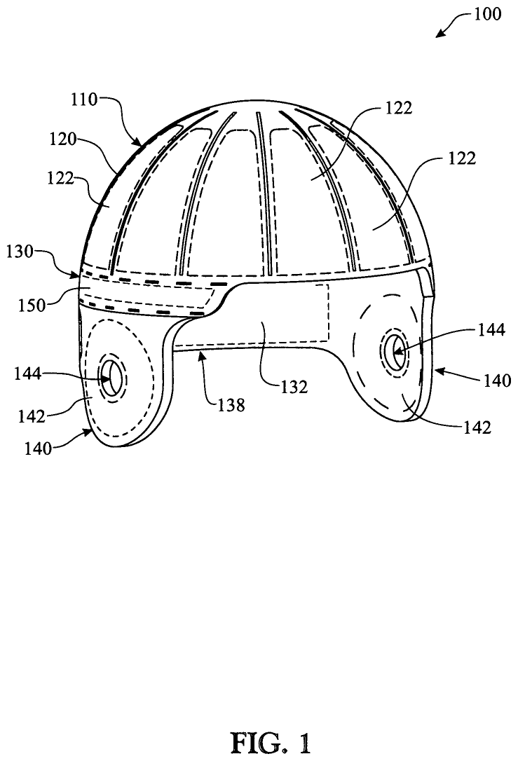

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, the present invention is directed toward a convenient and effective head-cooling cap that can be placed on a person's head immediately after a head injury to cool the person's head and thus help controlling swelling caused by the head injury.

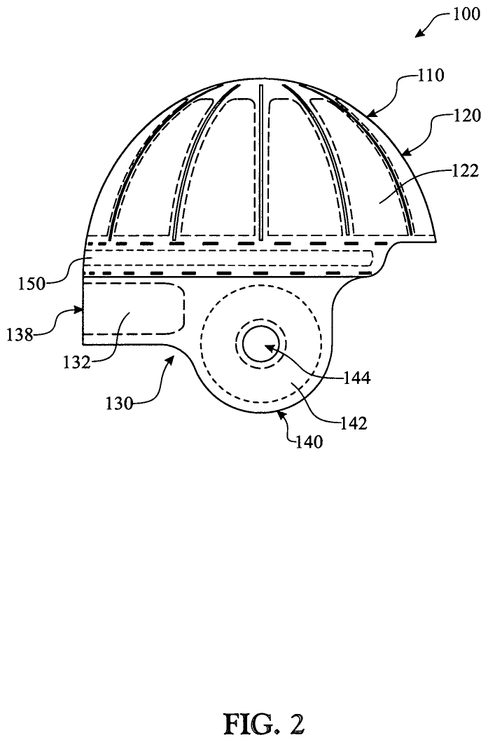

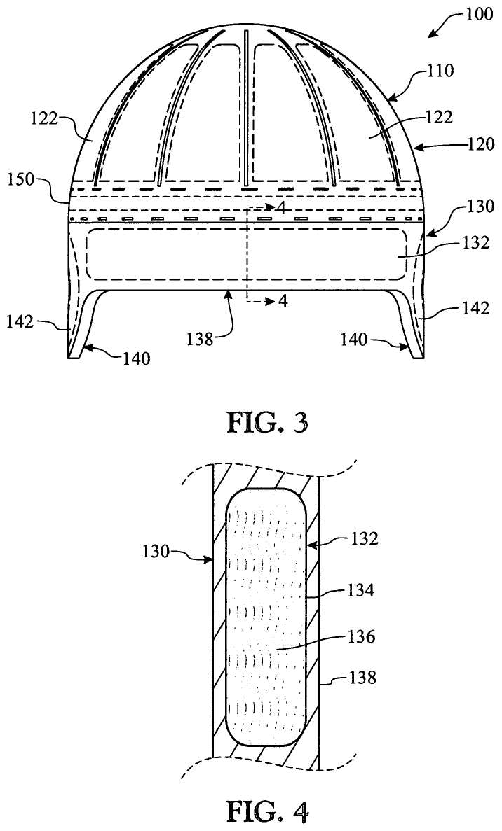

Referring initially to FIGS. 1 through 3, a head-cooling cap 100 is illustrated in accordance with a first exemplary embodiment of the present invention. As shown, the head-cooling cap 100 comprises a main body 110 including a top portion 120 and a side portion 130. The side portion 130 includes a rear, neck portion 138 and two opposed ear portions 140 protruding downward from the neck portion 138, where the neck portion 138 extends from one ear portion 140 to the other ear portion 140. The head-cooling cap 100 has a shape and size suitable to be placed on a user's head. The top portion 120 of the present embodiment is generally shaped as a semi-sphere, semi-ellipsoid or other concave body in order to fit onto the user's head. The side portion 130 is configured to cover the user's ears and back of the neck; the ears, in particular, are covered by the opposed ear portions 140. The main body 110 is generally made of one or more layers of a flexible, lightweight plastic.

The main body 110 further comprises a plurality of internal cavities formed within the main body 110, such as between two layers of flexible plastic forming the main body 110. Each internal cavity houses a cooling or refrigerating liquid or gel (hereinafter referred to as cooling substance). The cooling substance contained in each internal cavity forms a cooling element. The number and/or specific arrangement of the cooling elements can be selected based on the shape, size and desired cooling effect of the head-cooling cap 100. For instance, the present embodiment includes several top cooling elements 122 within the top portion 120, a rear side cooling element 132 within the rear portion 130 and one ear cooling element 142 within each ear portion 140. Alternative embodiments are contemplated in which the number of cooling elements comprised in each one of the top portion 120, the rear portion 130 and the ear portions 140 may vary.

The illustration of FIG. 4 presents an enlarged view of the rear side cooling element 132 of the neck portion 138 of the main body side portion 130. As shown, the rear side cooling element 132 is formed with an inner cavity 134 housing a cooling substance 136. The head-cooling cap 100 can be stored at freezing or cold temperatures for a sufficient amount of time to cause the cooling substance 136 to become adequately cold or frozen so that when the head-cooling cap 100 is placed on a user's head, the cooling substance 136 can cool the back of the neck and thus control swelling caused by any head injury.

In order not to block the hearing of a user, each ear cooling element 142 can be formed partially or fully surrounding a through hole 144 formed in the ear portion 140; for instance, each ear portion 140 of the head-cooling cap 100 of the embodiment includes only one ear cooling element 142 fully surrounding the respective through hole 144. The through hole 144 is configured to transversely align (i.e. be arranged in transverse registration) with a user's ear canal opening when wearing the head-cooling cap 100. The head-cooling cap 100 can be manufactured in different shapes and sizes for users to choose from. Alternatively, the head-cooling cap 100 can be made adjustable so as to adapt to different shapes and sizes of heads.

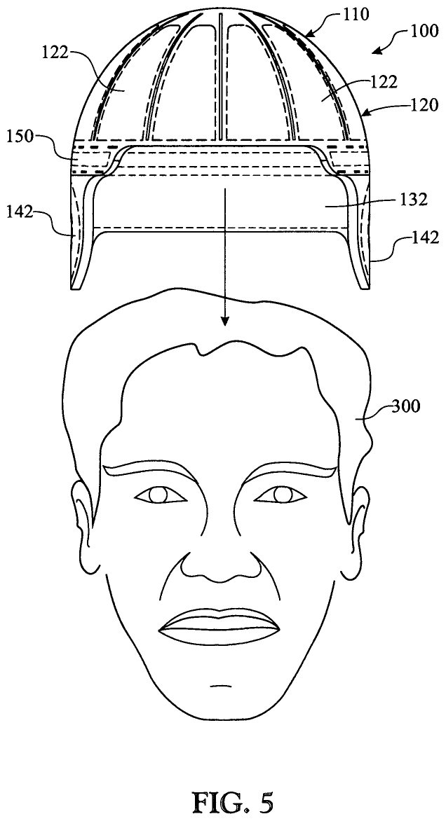

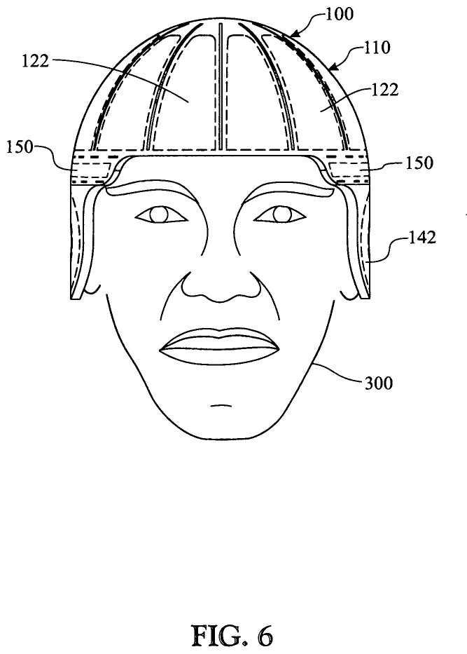

Referring now to FIGS. 5 and 6, the process of placing the head-cooling cap 100 according to FIGS. 1 through 3 onto a user's head 300 is illustrated. As shown in FIG. 5, the head-cooling cap 100 is being raised to right above the user's head 300 with the rear side cooling element 132 aiming toward the back of the neck of the user and the ear cooling elements 142 toward the ears of the user. As shown in FIG. 6, the head-cooling cap 100 is lowered down to snugly fit onto the user's head 300.

It is contemplated that the main body 110 can be integrally formed, as in the present embodiment, or formed of different parts attached to each other such as by stitching, heat welding, or removably (disconnectably) attachable fasteners such as hook-and-loop fasteners, snap fasteners, or the like.

The head-cooling cap 100 can include at least one elastic adjuster 150 to provide adjustment to the user's head 300. The elastic adjuster 150 of the present embodiment is arranged along the side portion 130, extending along the neck portion 138 and above the opposite ear portions 140. The elastic adjuster 150 can take the form, for instance, of an elastic band embedded within the flexible plastic materials forming the head-cooling cap main body 110.

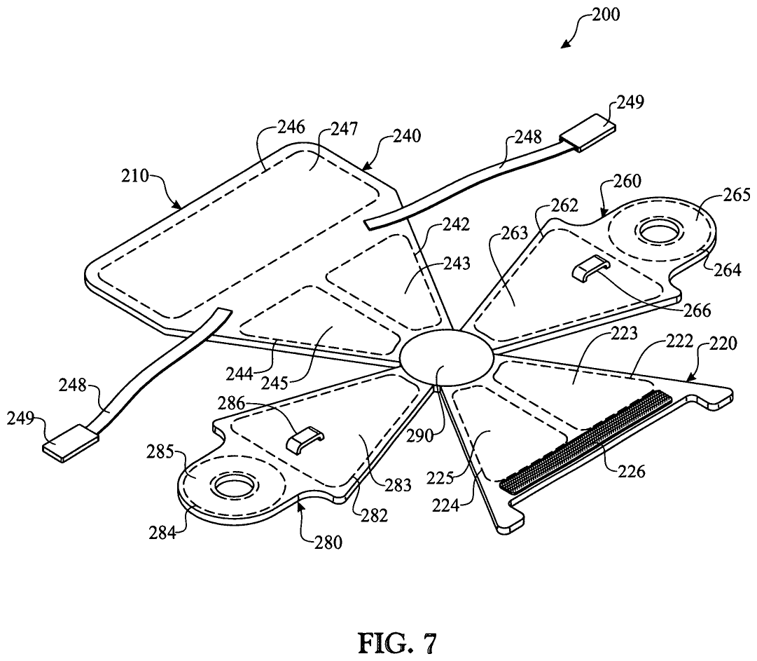

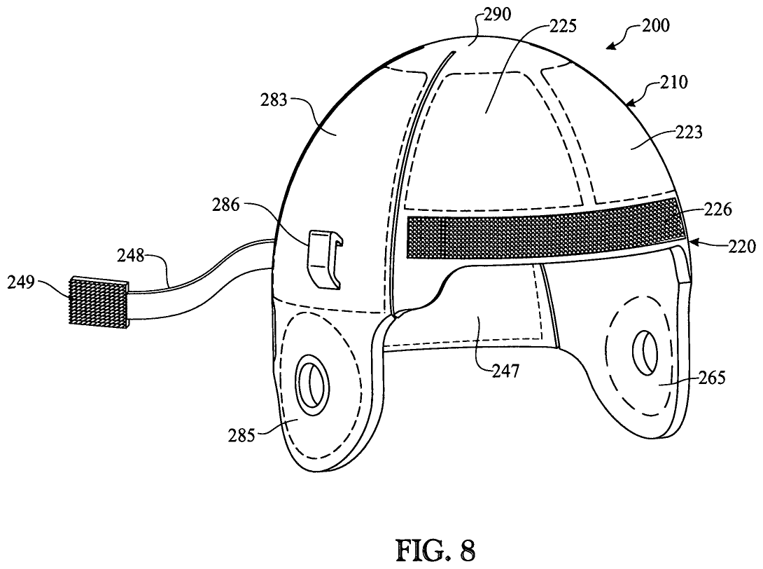

Referring now to FIGS. 7 and 8, a head-cooling cap 200 is illustrated in accordance with a second exemplary embodiment of the present invention. As shown, the head-cooling cap 200 is comprised of a generally planar main body 210 including four flexible portions: a front portion 220, a rear portion 240, and two lateral portions 260, 280. These flexible portions 220, 240, 260, 280 are connected at a crown portion 290, the flexible portions 220, 240, 260, 280 and preferably also the crown portion 290 being flexible and constructed in a flat format. Each flexible portion 220, 240, 260, 280 can be formed with one or more cooling elements provided by respective inner cavities housing a cooling substance such as a cooling gel. For example, the present embodiment is such that the front portion 220 is formed with two inner cavities 222, 224, each housing a cooling substance and thus forming a cooling element 223, 225 respectively; lateral portion 260 is formed with two inner cavities 262, 264 housing respective cooling substances and thus forming respective cooling elements 263, 265, of which a cooling element 265 is configured to be arranged over a person's ear; lateral portion 280 is formed with two inner cavities 282, 284 housing respective cooling substances and forming respective cooling elements 283, 285, of which a cooling element 285 is configured to be arranged over the opposite ear; the rear portion 240, in turn, is formed with three inner cavities 242, 244, 246, each housing a respective cooling substance and forming a respective cooling element 243, 245, 247, of which a cooling element 247 is configured to cool the back of the neck of a user. It is noted that the number of cooling elements as shown in FIG. 7 is for illustration purpose only and the present invention is not limited to the particular number of cooling elements shown.

The head-cooling cap 200 of the present embodiment can be deformed from the flat configuration shown in FIG. 7 to a three-dimensional configuration as shown in FIG. 8. In this three-dimensional configuration, proximal sections of the flexible portions 220, 240, 260, 280 form a main body of the head-cooling cap 200 presenting a cap-shape for fitting onto a user's head and comprising a plurality of cooling elements 223, 225, 243, 245, 263, 283. Distal sections of the lateral portions 260, 280 form ear portions of the head-cooling cap 200 for being arranged on a user's ears, the ear portions comprising ear cooling elements 265, 285. The rear portion 240 is longer than the front portion 220, and a distal section of rear portion 240 forms a neck portion of the head-cooling cap 200 for being arranged on the back of the user's neck, the neck portion comprising cooling element 247.

The head-cooling cap 200 can be kept in the three-dimensional configuration of FIG. 8 by disconnectably connectable fasteners, such as hook-and-loop fasteners, snap fasteners, or the like. Specifically, the rear portion 240 of the head-cooling cap 200 includes a pair of rear attachment straps 248, each having a strip of hooks 249 formed at an end thereof. The front portion 220 is formed with a strip of loops 226. The strips of hooks 249 and the strip of loop 226 together form the hook-and-loop fasteners that keep the cooling cap 200 in the three-dimensional format. In addition, as shown, the strip of loops 226 extend generally across the entire front portion 220, along the user's forehead; such arrangement allows the strips of hooks 249 to be attached to variable positions along the strip of loops 226 and thus provide for snug adjustment of the head-cooling cap 200 onto the user's head. The lateral portions 260, 280 of the present embodiment further include respective eyelets 266, 286 for the passing of the rear attachment straps 248 therethrough, contributing to keep the straps attached in place and to maintain the three-dimensional cap shape while the cap is placed on the user's head.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

1-20. (canceled)

21. A method for preventing or reducing brain injury to a boxer which comprises applying to the boxer a head-cooling cap for comprising:
a flexible main body configured to present a cap shape for fitting onto a user's head, said main body comprising a plurality of cooling elements, each cooling element of the plurality of cooling elements comprising a cooling substance housed within an inner cavity of the main body.

22. The method of claim 21, wherein the inner cavity of each cooling element is formed between two layers of flexible material that form the flexible main body.

23. The method of claim 21, wherein the cap further comprises at least one elastic adjuster to adjustably fit the flexible main body to a user's head.

24. The method of claim 21, wherein the main body of the head cooling cap comprises a concave top portion comprising at least one cooling element of the plurality of cooling elements, and a side portion extending from a bottom end of the concave top portion, the side portion comprising at least one cooling element of the plurality of cooling elements.

25. The method of claim 24, wherein the side portion of the head cooling cap comprises two opposed ear portions for covering a user's ear, each ear portion comprising at least one respective cooling element of the plurality of cooling elements.

26. The method of claim 25, wherein each ear portion of the head cooling cap comprises a through hole configured to be arranged in registration with an opening of an ear canal of a user wearing the head-cooling cap.

27. The method of claim 26, wherein each ear portion comprises a respective cooling element partially or fully surrounding the through hole.

28. The method of claim 24, wherein the side portion of the head cooling cap comprises a neck portion for covering a back of a neck of a user, the neck portion comprising at least one cooling element.

29. The method of claim 28, wherein the side portion further comprises two opposed ear portions for covering the user's ears, each ear portion protruding downward in regard to the neck portion and comprising at least one respective cooling element of the plurality of cooling elements, and further wherein the neck portion extends from one of the two opposed ear portions to the other of the two opposed ear portions.
